(19) United States
(12) Patent Application Publication
(10) Pub. No.: US 2017/0340636 A1
(43) Pub. Date: Nov. 30, 2017

(54) COMPOSITIONS AND METHODS FOR INHIBITION OF AUTOPHAGY

(71) Applicant: Board of Regents, The University of Texas System, Austin, TX (US)

(72) Inventors: Elizabeth J. Goldsmith, Dallas, TX (US); Alexander T. Piala, Dallas, TX (US); Malia B. Potts, Carrollton, TX (US)

(21) Appl. No.: 15/604,448

(22) Filed: May 24, 2017

Related U.S. Application Data
(60) Provisional application No. 62/341,937, filed on May 26, 2016.

(51) Int. Cl. A61K 31/517; A61K 31/428; A61K 31/427

(52) U.S. Cl. CPC A61K 31/517; A61K 31/427; A61K 31/428

(57) ABSTRACT

The present invention concerns compounds and their use to treat cell proliferative disorders including tumor growth and metastasis. Compounds of the present invention act as inhibitors of TAOK2 and modulators of autophagy, thereby functioning as anti-cancer or cancer-preventative therapeutic agents.

### REFERENCE TO RELATED APPLICATION

This application claims the benefit of U.S. Provisional Application No. 62/341,937, filed May 26, 2016, which is incorporated herein by reference in its entirety.

### STATEMENT OF GOVERNMENT RIGHTS

This invention was made with Government support under Grant No. RP100941 and Grant No. RP110595 awarded by the Cancer Prevention and Research Institute of Texas. The Government has certain rights in the invention.

### FIELD OF THE INVENTION

The present invention relates generally to the identification of compounds capable of inhibiting autophagy. More particularly, the invention relates to compounds exhibiting Thousand-And-One Kinase 2 (TAOK2) inhibitory activity, and related analogs useful in the treatment of cancer or prevention of cancer, and methods of treatment employing these compounds.

### BACKGROUND OF THE INVENTION

Autophagy is an evolutionarily conserved cell biological process through which cellular components are sequestered within a double membrane and delivered to the lysosome for degradation and recycling. Both basal and stress-induced autophagy can promote cell proliferation and tumor growth. Acute inhibition of autophagy in genetically engineered mouse models of cancer has demonstrated promising therapeutic benefit, but also systemic toxicity. A need therefore exists for new therapeutic tools for manipulating autophagy in a tissue-specific manner, which in turn requires a more thorough understanding of the signaling mechanisms that control autophagy during development and disease.

### SUMMARY OF THE INVENTION

In one aspect, the present invention provides methods of treating or preventing a cell proliferative disorder in a subject, comprising administering to said subject a therapeutically sufficient amount of a compound of Formula I:

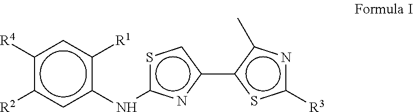

wherein: R¹ is selected from the group consisting of: OCH₃, OCH₂CH₃, H, CH₃, and CH₂CH₃; R² is selected from the group consisting of: OCH₃, Cl, H, CH₃, OH, NO₂, COCH₃, and COOH; R³ is selected from the group consisting of: NHCOCH₂CH₃, NHCOCH₃, NH₂, and NHCOC₆H₅; and R⁴ is selected from the group consisting of: H, SO₂NH₂, OH, N(CH₃)₂, Br, CH₃, NO₂, and NHCOCH₃. In some embodiments, R³ is NHCOCH₂CH₃ or NHCOCH₃. In further embodiments, the compound has Formula II:

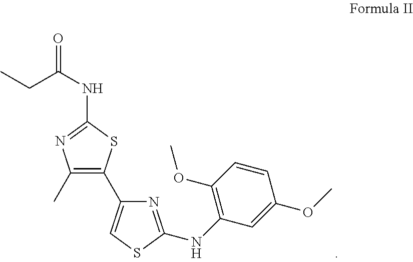 [SMILES: CCC(=O)Nc1nc(C)c(-c2csc(Nc3cc(OC)ccc3OC)n2)s1]

In certain embodiments of the cell proliferative disorder is selected from the group consisting of: non-small cell lung cancer or other lung or bronchus cancer, breast cancer, prostate cancer, colon and rectum cancer, bladder cancer, melanoma of the skin, non-Hodgkin lymphoma, thyroid cancer, kidney and renal pelvis cancer, leukemia, endometrial cancer, pancreatic cancer, ovarian cancer, osteosarcoma, and epstein barr virus. The compound may be administered via local, regional, systemic, or continual administration. In some embodiments, the subject is a human. In further embodiments, the methods provided by the invention further comprise providing to said subject a second therapy. In various embodiments, the second therapy may be provided prior to administering the compound, at the same time as the compound, or after administering the compound.

In another aspect, the invention provides a pharmaceutical composition comprising a compound dispersed in a pharmaceutically acceptable carrier, buffer or diluent, wherein the compound has Formula I:

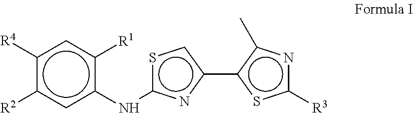

wherein: R¹ is selected from the group consisting of: OCH₃, OCH₂CH₃, H, CH₃, and CH₂CH₃; R² is selected from the group consisting of: OCH₃, Cl, H, CH₃, OH, NO₂, COCH₃, and COOH; R³ is selected from the group consisting of: NHCOCH₂CH₃, NHCOCH₃, NH₂, and NHCOC₆H₅; and R⁴ is selected from the group consisting of: H, SO₂NH₂, OH, N(CH₃)₂, Br, CH₃, NO₂, and NHCOCH₃.

In a further embodiment, the present invention provides methods of treating or preventing a cell proliferative disorder in a subject, comprising administering to said subject a therapeutically sufficient amount of a compound of Formula III:

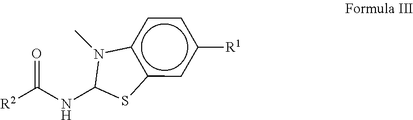

wherein: R¹ is SO₂CH₃; and R² comprises a cycloalkyl or aromatic group. In some embodiments, R² has Formula IV:

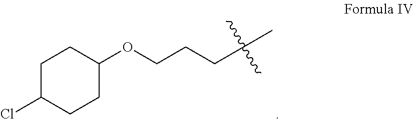 [SMILES: *CCCOC1CCC(Cl)CC1]

In other embodiments, the compound has Formula V:

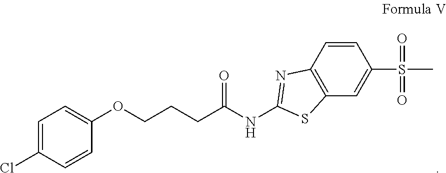 [SMILES: CS(=O)(=O)c1ccc2nc(NC(=O)CCCOc3ccc(Cl)cc3)sc2c1]

In certain embodiments of the cell proliferative disorder is selected from the group consisting of: non-small cell lung cancer or other lung or bronchus cancer, breast cancer, prostate cancer, colon and rectum cancer, bladder cancer, melanoma of the skin, non-Hodgkin lymphoma, thyroid cancer, kidney and renal pelvis cancer, leukemia, endometrial cancer, pancreatic cancer, ovarian cancer, osteosarcoma, and epstein barr virus. The compound may be administered via local, regional, systemic, or continual administration. In some embodiments, the subject is a human. In further embodiments, the methods provided by the invention further comprise providing to said subject a second therapy. In various embodiments, the second therapy may be provided prior to administering the compound, at the same time as the compound, or after administering the compound.

In another aspect, the invention provides a pharmaceutical composition comprising a compound dispersed in a pharmaceutically acceptable carrier, buffer or diluent, wherein the compound has Formula III:

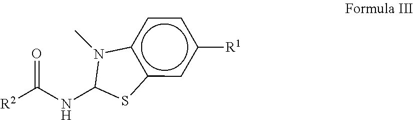

wherein: R¹ is SO₂CH₃; and R² comprises a cycloalkyl or aromatic group.

In a further aspect, the invention provides methods of treating or preventing a cell proliferative disorder in a subject, comprising administering to said subject a therapeutically sufficient amount of a compound of Formula VI:

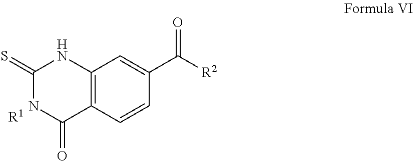

wherein: R¹ comprises an ether or a flat ring system; and R² comprises a five- or six-member ring system. In certain embodiments, R¹ has the formula of Formula VII:

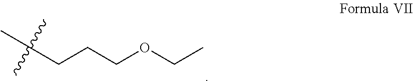 [SMILES: *CCCOCC]

In other embodiments, R² has the formula of Formula VIII:

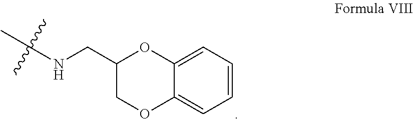 [SMILES: *NCC1COc2ccccc2O1]

In further embodiments, the compound has the formula of Formula IX:

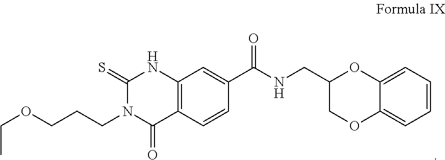 [SMILES: CCOCCCn1c(=S)[nH]c2cc(C(=O)NCC3COc4ccccc4O3)ccc2c1=O]

In certain embodiments of the cell proliferative disorder is selected from the group consisting of: non-small cell lung cancer or other lung or bronchus cancer, breast cancer, prostate cancer, colon and rectum cancer, bladder cancer, melanoma of the skin, non-Hodgkin lymphoma, thyroid cancer, kidney and renal pelvis cancer, leukemia, endometrial cancer, pancreatic cancer, ovarian cancer, osteosarcoma, and epstein barr virus. The compound may be administered via local, regional, systemic, or continual administration. In some embodiments, the subject is a human. In further embodiments, the methods provided by the invention further comprise providing to said subject a second therapy. In various embodiments, the second therapy may be provided prior to administering the compound, at the same time as the compound, or after administering the compound.

In another aspect, the invention provides a pharmaceutical composition comprising a compound dispersed in a pharmaceutically acceptable carrier, buffer or diluent, wherein the compound has Formula III:

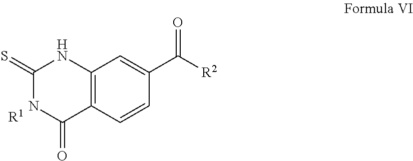

wherein: R¹ comprises an ether or a flat ring system; and R² comprises a five- or six-member ring system.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D shows high throughput screen results. FIG. 1A) A rank-ordered graph of the Z-score of each compound tested in the high-throughput screen. The lower dotted line shows the average. The upper dotted line is 3σ. Compounds above 3σ were chosen for subsequent screening (grey). FIG. 1B) Z′ factor for each plate of the screen is shown. The average Z′ factor across the screen is shown as a solid dotted line. FIG. 1C) % inhibition in the confirmation screen vs. % inhibition in the original HTS screen is plotted to show consistency between screens. FIG. 1D) The % inhibition of each compound is plotted at 1 μM (light gray), 3.3 μM (black), and 10 μM (dark gray).

FIG. 2 shows TAOK2 inhibitors discovered from 200 k compound library.1 Thirteen previously unidentified TAOK2 inhibitors are disclosed. IC50 values determined from the screen ranged from 0.3 to 6 μM.

FIG. 3 shows a specificity screen for TAOK2 inhibitors discovered from 200 k compound library. Of the 13 inhibitors, 3 were tested for cross-reactivity against 45 off-target kinases. Kinases showing inhibition higher that 50% (dotted line) are considered significant.

FIGS. 4A-4B demonstrates that TAOK2 inhibition prevents cellular autophagy. FIG. 4A) U2OS cells stably expressing the GFP-LC3 fusion transgene were treated with the indicated compound at 50 μM concentration for 24 hours before GFP fluorescence was measured by flow cytometry. The median fluorescent value from 20,000 cells per condition was normalized to that of vehicle alone (DMSO) within a given experiment. The mean+SD of >2 independent experiments is shown. ****, p<0.0001; ***, p<0.001; **, p<0.01; ns, p>0.05 versus vehicle alone (DMSO) by unpaired t test. FIG. 4B) TAOK2 inhibitors induce accumulation of p62. U2OS GFP-LC3 cells were treated with the indicated compounds at 50 μM concentration, or DMSO alone, for 24 hours. Lysates were collected and analyzed by western blot using the indicated antibodies. XPB was used as a loading control.

FIGS. 5A-5C shows homologs of compound SW034538, SW172006, and SW083688 in the HTS. Compounds with similar structure to (FIG. 5A) SW034538, (FIG. 5B) SW172006, and (FIG. 5C) SW083688 were tested in the HTS screen. Missing IC₅₀ values represent compounds that did not meet the 3 sigma threshold for the confirmation screen.

### DETAILED DESCRIPTION

Autophagy is a cellular process through which cellular components are targeted for degradation and recycling. Although this process has been linked to cell proliferation, hypertrophy, and hyperplasia, including tumor growth and metastasis, acute inhibition of autophagy results in systemic toxicity. A need therefore exists for new therapeutic tools for manipulating autophagy in a tissue-specific manner. The Mitogen Activated Protein Kinase Kinase Kinase (MAP3K) TAOK2 is an activator of p38 MAP kinase cascade that is upregulated in response to environmental stresses, and has been identified as a potential modulator of autophagy.

In order to address the need for new therapeutic agents for modulation of autophagy, the present invention provides small molecule compounds which inhibit the kinase activity of TAOK2. Structure-activity relationship studies (SAR) studies have been conducted based on the identification of these TAOK2 inhibitors to identify structural analogs exhibiting similar activity. The compounds provided by the invention are useful in the manipulation of autophagy, and are therefore useful in the treatment and prevention of cell-proliferative disorders including cancer growth and metastasis.

I. Compounds with TAOK2 Inhibitory Activity

The compounds provided by the invention have utility in the therapeutic modulation of cell proliferation, hypertrophy, and hyperplasia, including tumor growth and metastasis. For example, the compounds of the present invention are useful in the prevention or treatment of non-small cell lung cancer, osteosarcoma, ovarian cancer, breast cancer, colon cancer, or epstein barr virus. In addition, the compounds provided by the present invention are useful in the amelioration of side-effects of existing cancer therapeutic agents. For example, modulation of autophagy in a cell-type specific manner may prevent or treat ototoxicity resulting from treatment with cisplatin. Exemplary molecules according to the present invention which exhibit TAOK2 inhibitory activity are shown below. Methods for using the compounds of the invention in methods for treatment of cell-proliferative disorders are further provided.

One aspect of the invention comprises methods of treating or preventing cell proliferative disorders comprising administering to a subject compounds of Formula I:

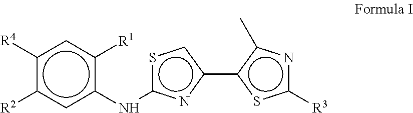

In certain aspects of the invention, the chemical structures shown in Formula I may be defined as follows: R¹ is selected from the group consisting of: OCH₃, OCH₂CH₃, H, CH₃, and CH₂CH₃; R² is selected from the group consisting of: OCH₃, Cl, H, CH₃, OH, NO₂, COCH₃, and COOH; R³ is selected from the group consisting of: NHCOCH₂CH₃, NHCOCH₃, NH₂, and NHCOC₆H₅; and R⁴ is selected from the group consisting of: H, SO₂NH₂, OH, N(CH₃)₂, Br, CH₃, NO₂, and NHCOCH₃.

In certain embodiments of the invention, R³ is NHCOCH₂CH₃ or NHCOCH₃. For example in specific embodiments, R³ is NHCOCH₃.

Further embodiments of the invention comprise administering compounds of Formula II:

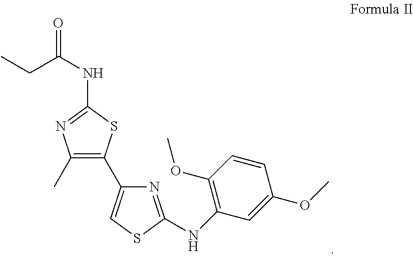 [SMILES: CCC(=O)Nc1nc(C)c(-c2csc(Nc3cc(OC)ccc3OC)n2)s1]

Another aspect of the invention comprises methods of treating or preventing cell proliferative disorders comprising administering to a subject compounds of Formula III:

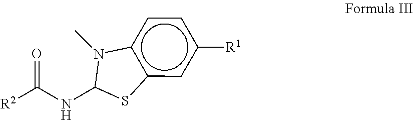

In certain aspects of the invention, the chemical structures shown in Formula II may be defined as follows: R¹ is SO₂CH₃; and R² comprises a cycloalkyl or aromatic group. In specific embodiments, R² has Formula IV:

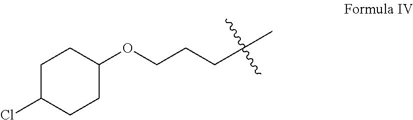 [SMILES: *CCCOC1CCC(Cl)CC1]

Further embodiments of the invention comprise administering compounds having Formula V:

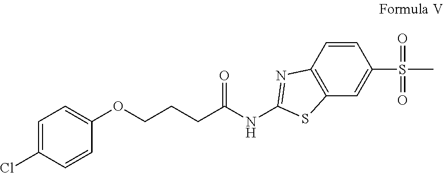 [SMILES: CS(=O)(=O)c1ccc2nc(NC(=O)CCCOc3ccc(Cl)cc3)sc2c1]

A further aspect of the invention comprises methods of treating or preventing cell proliferative disorders comprising administering to a subject compounds of Formula VI:

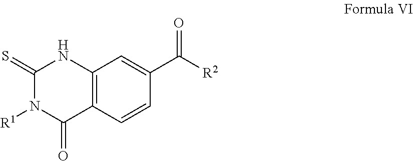

In certain aspects of the invention, the chemical structures shown in Formula III may be defined as follows: R¹ comprises an ether or a flat ring system; and R² comprises a five- or six-member ring system.

In specific embodiments, R¹ has the formula of Formula VII:

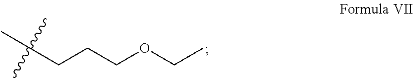 [SMILES: *CCCOCC]

or R² has the formula of Formula VIII:

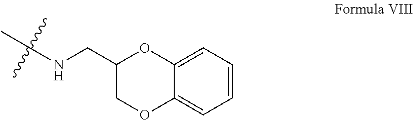 [SMILES: *NCC1COc2ccccc2O1]

Further embodiments of the invention comprise administering compounds having the formula of Formula IX:

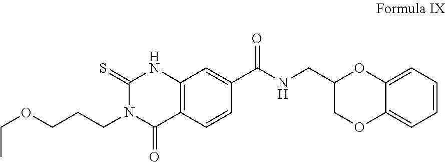 [SMILES: CCOCCCn1c(=S)[nH]c2cc(C(=O)NCC3COc4ccccc4O3)ccc2c1=O]

Pharmaceutical compositions comprising a compound provided by the invention dispersed in a pharmaceutically acceptable carrier, buffer or diluent are further provided. Other aspects of the invention include pharmaceutically acceptable salts, hydrates, tautomers, and optical isomers of the compounds described above and throughout this application.

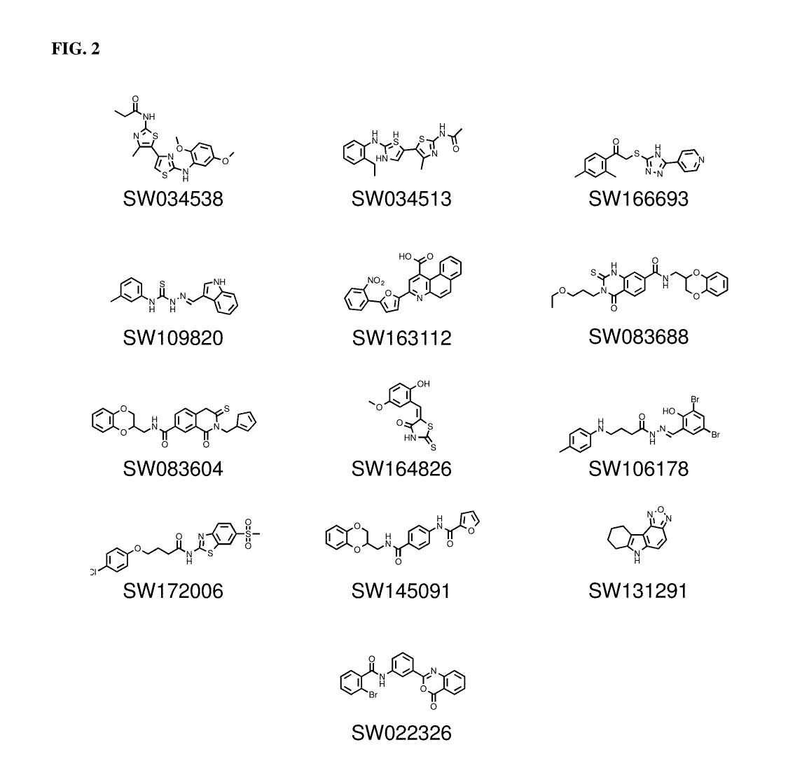

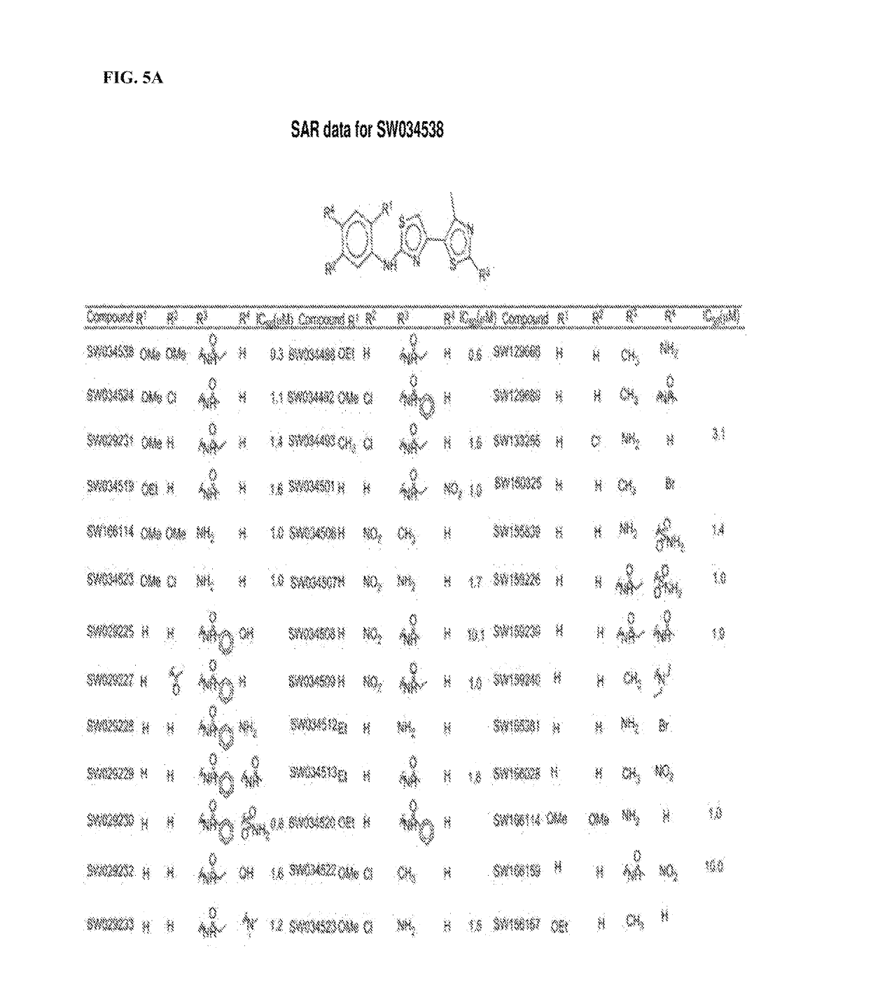

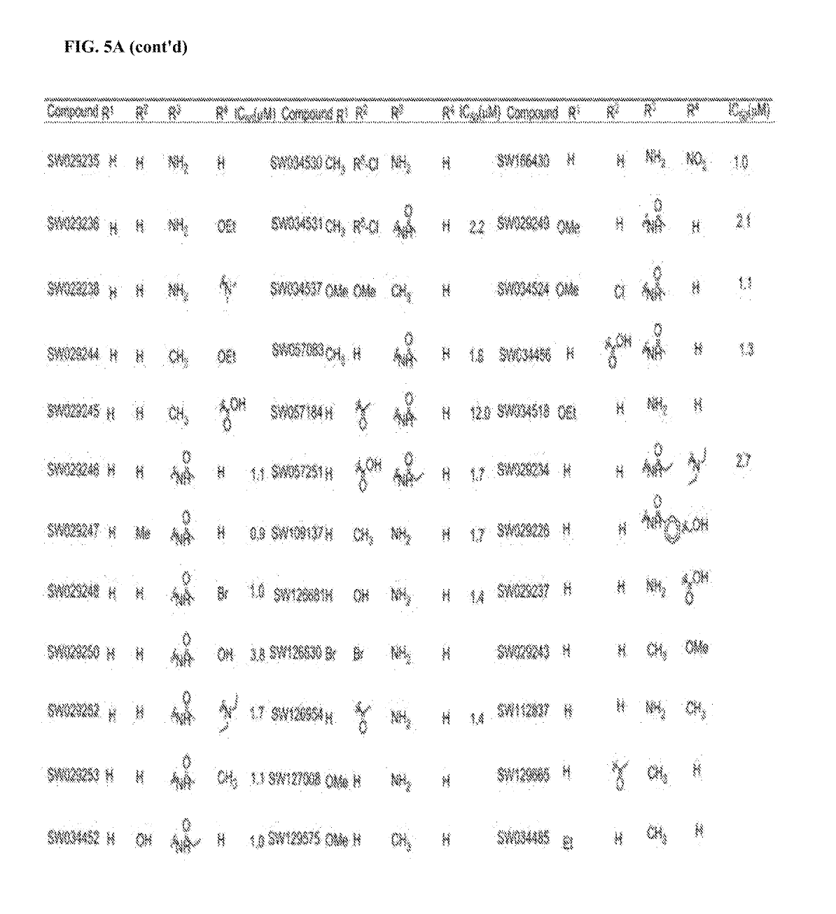

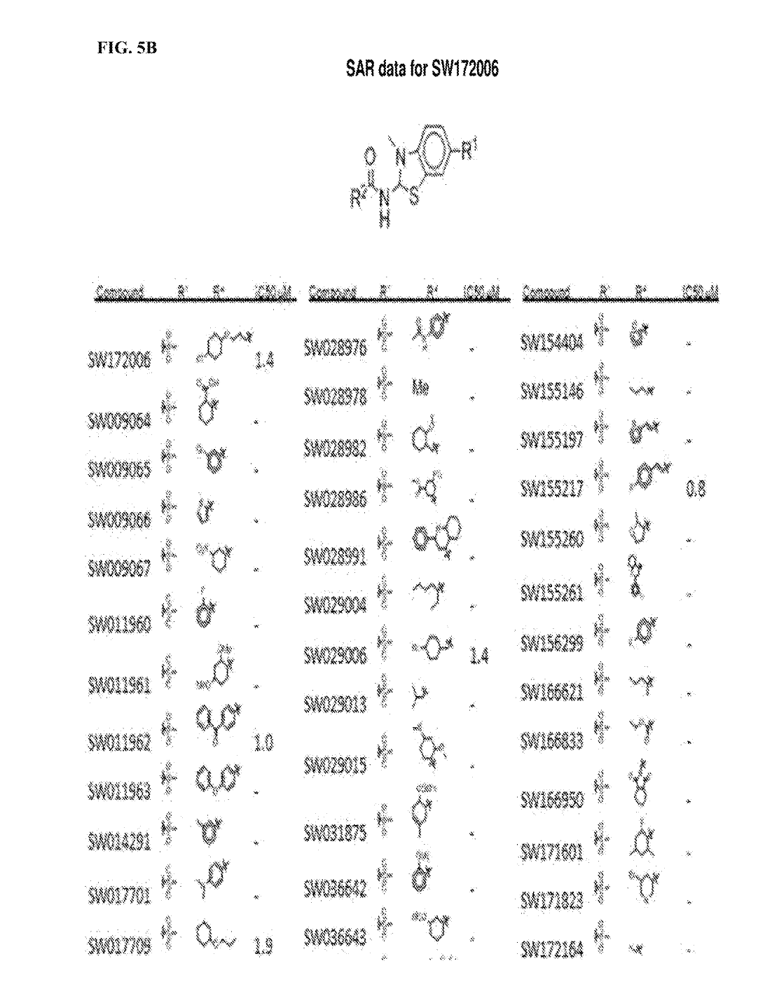

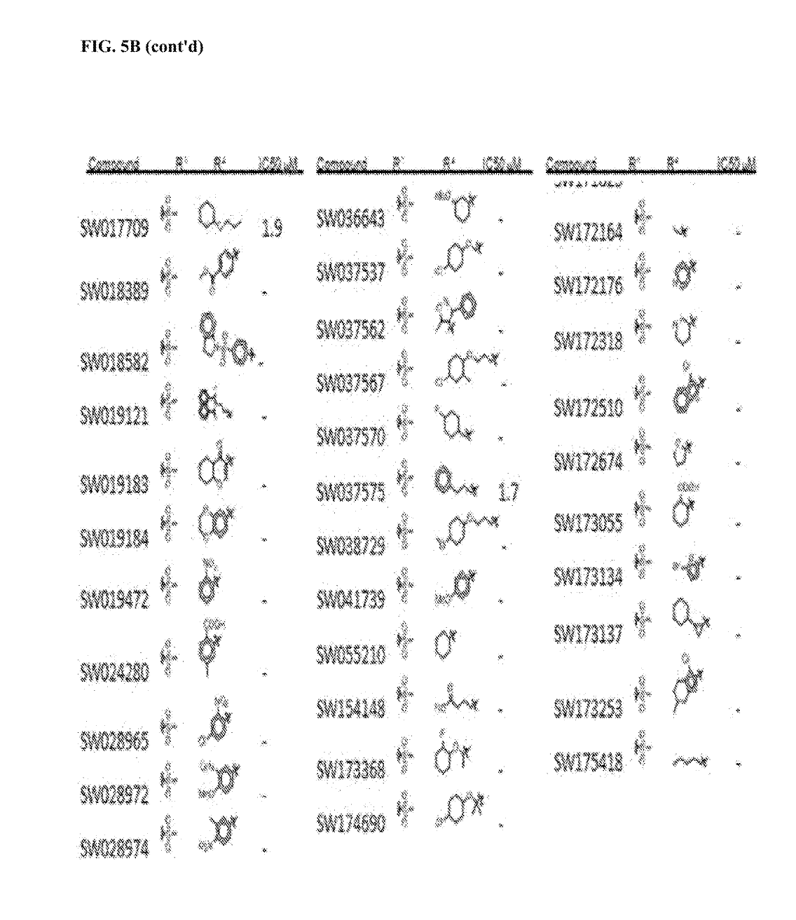

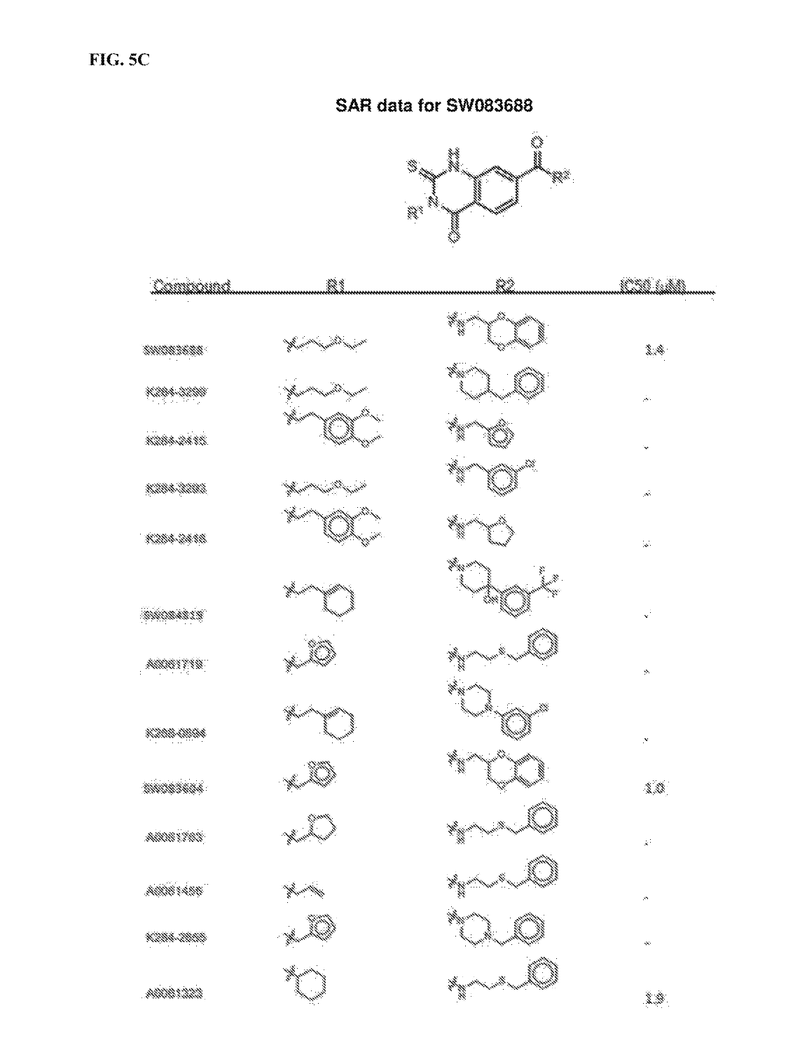

FIGS. 2 and 5 show specific examples of compounds provided for use in the methods of the invention.

Another aspect of the present invention concerns a method of treating a disease, including non-small cell lung cancer or other lung or bronchus cancer, breast cancer, prostate cancer, colon and rectum cancer, bladder cancer, melanoma of the skin, non-Hodgkin lymphoma, thyroid cancer, kidney and renal pelvis cancer, leukemia, endometrial cancer, pancreatic cancer, ovarian cancer, osteosarcoma, and epstein barr virus, comprising administering a therapeutically relevant amount of a first compound of the present invention to a subject. In some embodiments, the first compound is of Formulas I, II, III, V, VI, IX, or the compounds listed in FIGS. 2 and 5. The subject may be a mammal, and the mammal may be a human. The first compound may be comprised in a pharmaceutically acceptable excipient, diluent, or vehicle. The first compound may be administered in combination with a therapeutically relevant amount of a second compound. The first compound may be administered in combination with a surgery, a radiation therapy, or a gene therapy.

Any embodiment discussed herein with respect to one aspect of the invention applies to other aspects of the invention as well, unless specifically noted.

### II. Chemical Definitions

An “alkane” refers to an acyclic branched or unbranched hydrocarbon, in many cases having the general formula CₙH₂ₙ₊₂. An “alkyl” refers to a univalent group derived from an alkane by removal of a hydrogen atom from any carbon atom thus having the formula —CₙH₂ₙ₊₁ in many cases. Alkyl groups, either straight-chained or branched chained, may be substituted with additional acyclic alkyl, cycloalkyl, or cyclic alkyl groups. The alkyl group may be heteroatom-substituted or heteroatom-unsubstituted. Preferably, the alkyl group has 1 to 12 carbons. More preferably, it is a lower alkyl having 1 to 7 carbons, more preferably 1 to 4 carbons. An upper alkyl has 8 or more carbon atoms. A “divalent alkyl” refers to a divalent group derived from an alkane by removal of two hydrogen atoms from either the same carbon atom (e.g. methylene, ethylidene, propylidene) or from different carbon atoms (e.g. —C₂H₄—).

A “cycloalkane” refers to a saturated monocyclic hydrocarbon with or without side chains.

As used herein, the term “alkoxy” refers to an alkyl group bonded to an oxygen; having the formula —OR, wherein R represents an alkyl group. For example, “methoxy” or “OMe” means —O—CH₃, and refers to an oxygen atom bound to a methyl group. The term “ethoxy” or “OEt” means —OCH₂CH₃, and refers to an oxygen bound to an ethyl group.

As used herein, the term “halo” designates F, Cl, Br or I. The term “nitro” means —NO₂.

As used herein, the term “carboxamide” refers to a functional group having the formula RC(O)NR′₂. For example, “ethanamide” or “acetamide” refers to a functional group having the formula CH₃CONH, and “propanamide” refers to a functional group having the formula CH₃CH₂CONH—. Benzamide refers to a functional group having a formula of C₇H₇NO—.

As used herein, the term “sulfonamide” refers to a functional group comprising a sulfonyl group connected to an amine group, for example a functional group having the formula SO₂NH₂.

The term “acetyl” refers to a carbonyl bonded to a methyl group, having the formula —COCH3. The term “carboxyl” refers to a functional group having this structure —C(O)OH.

The term “heteroatom-substituted,” when used to modify a class of organic radicals (e.g. alkyl, aryl, acyl, etc.), means that one, or more than one, hydrogen atom of that radical has been replaced by a heteroatom, or a heteroatom containing group. Examples of heteroatoms and heteroatom containing groups include: hydroxy, cyano, alkoxy, ═O, ═S, —NO₂, —N(CH₃)₂, amino, or —SH.

The term “ring system” refers to any homocyclic or heterocyclic ring structure comprising one or more rings, for example cycloalkane or aromatic functional groups.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like. Other suitable salts are known to one of ordinary skill in the art.

Suitable pharmaceutically acceptable salts may also be formed by reacting the agents of the invention with an organic base such as methylamine, ethylamine, ethanolamine, lysine, ornithine and the like. Other suitable salts are known to one of ordinary skill in the art.

Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

It should be recognized that the particular anion or cation forming a part of any salt of this invention is not critical, so long as the salt, as a whole, is pharmacologically acceptable and as long as the anion or cation does not contribute undesired qualities or effects. Further, additional pharmaceutically acceptable salts are known to those skilled in the art, and may be used within the scope of the invention. Additional examples of pharmaceutically acceptable salts and their methods of preparation and use are presented in Pharmaceutical Salts: Properties, Selection and Use-A Handbook (2002), which is incorporated herein by reference.

### III. TAOK2, Autophagy, and Cell Proliferative Disease

TAOK2 is a sterile 20-like kinase and a member of the germinal-center kinase-like kinase family noted for their N-terminal positioning of the kinase domain. Its major isoform is 1235 residues and is ubiquitously expressed. Activation of TAOK2 results in stalling of cell division at the G2/M checkpoint. The knockdown of kinases downstream of TAOK2 has been shown to sensitize cells to UV insult. 30% of pancreatic cancers have mutations in the coding region of this kinase, highlighting its potential therapeutic importance.

TAOK2 has further been implicated as a regulator of autophagy. Autophagy is an evolutionarily conserved cell biological process through which cellular components are sequestered within a double membrane and delivered to the lysosome for degradation and recycling. Both basal and stress-induced autophagy can play cytoprotective, growth-enhancing, and tumor-promoting roles. Acute inhibition of autophagy in genetically engineered mouse models of cancer has demonstrated promising therapeutic benefit, but also systemic toxicity. This underscores the need to develop therapeutic tools for manipulating autophagy in a tissue-specific manner, which in turn requires a more thorough understanding of the signaling mechanisms that control autophagy during development and disease.

The present invention addresses this need in the art by providing TAOK2 inhibitory molecules capable of modulating autophagy. In certain embodiments, compounds and methods of the present invention may be used to treat a wide variety of cell proliferative disorders including cancer or conditions associated with cancer therapy.

### IV. Therapeutic Use of TAOK2 Inhibitors

Based on TAOK2 inhibitory bioactivity in vitro and in vivo, it is anticipated that compounds of the present invention may be used alone or in combination with other therapeutic agents in the treatment of the following conditions:

### Monotherapy

Acute inhibition of autophagy (via ATG7 ablation) has therapeutic benefit in a mouse model of non-small cell lung cancer. As described herein, the TAOK2 inhibitors of the present invention are capable of inhibiting autophagy, and are therefore useful in the treatment of cancer or other cell proliferative diseases. The compounds of the present invention may be used alone or in combination with other agents to treat or prevent cancer, including non-small cell lung cancer or other lung or bronchus cancer, breast cancer, prostate cancer, colon and rectum cancer, bladder cancer, melanoma of the skin, non-Hodgkin lymphoma, thyroid cancer, kidney and renal pelvis cancer, leukemia, endometrial cancer, pancreatic cancer, ovarian cancer, and osteosarcoma. The compounds provided by the present invention are further useful in treating Epstein Barr virus.

### Combination Therapy

Autophagy contributes to radioresistance and chemoresistance in osteosarcoma, and inhibiting autophagy sensitizes osteosarcoma cells to radiation and chemotherapeutics. Treating ovarian cancer cells with cisplatin causes Ambral upregulation and induction of autophagy, and this likely contributes to resistance, because depleting Ambral by shRNA increased cisplatin sensitivity. Thus, the compounds of the present invention may be used in combination with other compounds to treat or prevent cancer, including non-small cell lung cancer or other lung or bronchus cancer, breast cancer, prostate cancer, colon and rectum cancer, bladder cancer, melanoma of the skin, non-Hodgkin lymphoma, thyroid cancer, kidney and renal pelvis cancer, leukemia, endometrial cancer, pancreatic cancer, ovarian cancer, and osteosarcoma. The compounds provided by the present invention are further useful in combination therapies for the treatment of Epstein Barr virus.

### Cancer Prevention

ATG7 ablation (which blocks autophagy) in the intestinal epithelium protects APC+/− mice from developing precancerous lesions through a mechanism that requires gut microbiota and CD8+ T cells. FIP200 deletion in the mammary pad inhibits autophagy and protects against mmtv-PyMT-driven tumorigenesis. In the PALB2 breast cancer model, loss of one allele of beclin 1 reduces autophagy and delays tumor initiation. These cumulative results suggest that pharmacological inhibition of autophagy, such as provided by the TAOK2 inhibitory compounds of the present invention, are useful in the prevention of breast or colon cancer in high-risk patients such as those harboring germline mutations in APC, BRCA1, or BRCA2 as well as survivors of childhood cancer. Furthermore, some cancer-causing viral infections may be treatable by autophagy inhibition. Epstein Barr virus utilizes the early steps of macroautophagy for infectious viral production, and inhibition of autophagy as provided by the TAOK2 inhibitors of the invention dampens infectious particle production.

### Amelioration of Side Effects

Development of therapeutic strategies for modulating autophagy in cell-type-specific fashion is further useful in ameliorating side-effects of current anti-cancer therapies. For example, autophagy may contribute to ototoxicity in response to cisplatin. The TAOK2 inhibitors of the present invention therefore find further utility in ameliorating the side-effects of existing anti-cancer therapies.

### V. Pharmaceutical Compositions

The compounds of the present invention can be administered to interfere with autophagy and function to treat or prevent cell proliferative diseases such as cancer, by any method that allows contact of the active ingredient with the agent's site of action in a cell. They can be administered by any conventional methods available for use in conjunction with pharmaceuticals, either as individual therapeutically active ingredients or in a combination of therapeutically active ingredients. They can be administered alone but are generally administered with a pharmaceutically acceptable carrier selected on the basis of the selected route of administration and standard pharmaceutical practice.

Aqueous compositions of the present invention will have an effective amount of the compounds to function as anti-cancer or cancer-preventative agents. Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic or other undesirable reaction when administered to an animal, including a human. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer or cancer-preventative agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including creams, lotions, mouthwashes, inhalants, lipid carriers, liposomes and the like.

### Parenteral Administration

The active compounds provided by the present invention will often be formulated for parenteral administration, e.g., formulated for injection via the intravenous, intramuscular, subcutaneous, or even intraperitoneal routes. The preparation of an aqueous composition that contains a TAOK2 inhibitor of the present invention as an active ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

In some forms, it will be desirable to formulate the compounds in salt form, generally to improve the solubility and bioavailability and to provide an active drug form more readily assimilated. As used herein, the term “pharmaceutically acceptable salt” refers to compounds which are formed from acidifying a TAOK2 inhibitor solution of the invention with suitable physiologically tolerated acids. Suitable physiologically tolerated acids are organic and inorganic acids, such as hydrochloric acid, sulfuric acid, phosphoric acid, acetic acid, citric acid, oxalic acid, malonic acid, salicylic acid, maleic acid, methane sulfonic acid, isothionic acid, lactic acid, gluconic acid, glucuronic acid, amidosulfuric acid, benzoic acid, tartaric acid and pamoaic acid. Typically, such salt forms of the active compound will be provided or mixed prior to use.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds provided by the invention may be formulated into a composition in a neutral or salt form. Pharmaceutically acceptable salts, include the acid addition salts and which are formed with inorganic acids such as, for example, hydrochloric or phosphoric acids, or such organic acids as acetic, oxalic, tartaric, mandelic, and the like.

The compounds of the present invention may also be formulated into a composition comprising liposomes or any other lipid carrier. Liposomes include: multivesicular liposomes, multilamellar liposomes, and unilamellar liposomes.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in creams and lotions. These forms may be used for treating skin-associated diseases.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this regard, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

### Oral Administration

In certain embodiments, active compounds provided by the invention may be administered orally. This is contemplated for agents which are generally resistant, or have been rendered resistant, to proteolysis by digestive enzymes. Such compounds are contemplated to include all those compounds, or drugs, that are available in tablet form from the manufacturer and derivatives and analogues thereof.

For oral administration, the active compounds of the invention may be administered, for example, with an inert diluent or with an assimilable edible carrier, or they may be enclosed in hard or soft shell gelatin capsule, or compressed into tablets, or incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of the unit. The amount of active compounds in such therapeutically useful compositions is such that a suitable dosage will be obtained.

The tablets, troches, pills, capsules and the like may also contain the following: a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Upon formulation, the compounds will be administered in a manner compatible with the dosage formulation and in such amount as is therapeutically effective. The formulations are easily administered in a variety of dosage forms, such as those described below in specific examples.

### VI. Therapies

In the context of the present invention, it is contemplated that the TAOK2 inhibitor compounds provided by the invention could be used in combination with new or existing pharmaceutical agents, surgery, chemotherapy, radiotherapy, and/or gene therapy. In particular embodiments, the TAOK2 inhibitory compounds of the present invention are effective in increasing the efficacy of paclitaxel or cisplatin when used in combination therapies.

An “effective amount” or a “therapeutically relevant amount” are those amounts of a compound sufficient to produce a therapeutic benefit (e.g., effective to function as anti-cancer or cancer preventative treatment). An effective amount, in the context of treating a subject, is sufficient to produce a therapeutic benefit. The term “therapeutic benefit” as used herein refers to anything that promotes or enhances the well-being of the subject with respect to the medical treatment of the subject's disease. A nonexhaustive list of examples of therapeutic benefits includes extension of the subject's life by any period of time; decrease or delay in development of disease; decrease in hypertension; decrease in inflammation; decrease in cell growth or proliferation; and/or a decrease in pain to the subject that can be attributed to the subject's condition.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

The terms “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

As used herein, the term “subject” is intended to include living organisms in which certain conditions as described herein can occur. Examples include humans, monkeys, cows, sheep, goats, dogs, cats, mice, rats, and transgenic species thereof. In a preferred embodiment, the subject is a primate. In an even more preferred embodiment, the primate is a human. Other examples of subjects include experimental animals such as mice, rats, dogs, cats, goats, sheep, pigs, and cows. The experimental animal can be an animal model for a disorder, e.g., a transgenic mouse or rat exhibiting hypertension or metabolic syndrome. A subject can be a human suffering from a disease, for example a cardiovascular, renal, or metabolic disease, or cancer.

As used herein, the term “IC₅₀” refers to an inhibitory dose which is 50% of the maximum response obtained.

Other objects, features and advantages of the present invention will become apparent from this detailed description and examples provided below. It should be understood, however, that the detailed description and any specific examples provided, while indicating specific embodiments of the invention, are given by way of illustration only, since various changes and modifications within the spirit and scope of the invention will become apparent to those skilled in the art from this detailed description.

### EXAMPLES

### Example 1

### Methods for High-Throughput Screening and Binding Assays

### Isolation and Purification of TAOK2

TAOK2 was purified as reported in Zhou, et al. Structure, 12:1891, 2004.

### HTS Screen Conditions

TAOK2 activity was screened in a high-throughput fashion using Kinase-Glo (Promega). A 20 μL, solution of 50 mM HEPES pH 7.4, 50 mM MgCl₂, 1.27 mg/ml MBP and 0.005 mg/ml TAOK2 was added to each well of a 384 well Corning plate using a Biotek MultiFlo dispenser. 0.2 μl of compound (columns 3 to 22, 50 μM), DMSO negative control (columns 2, 23, and 24), or staurosporine positive control (column 1, 43.4 μM) were then added to each well with a Biomek Liquid handler. 10 μl of 50 μg/ml ATP solution was added to each well with a Labsystems Multidrop384. The plates were incubated at RT for 100 minutes. 20 μl Kinase Glo (Promega) was added to each well, and allowed to incubate for 10minutes under agitation. Luminescence was read on an Envision Multilabel plate reader.

### Differential Scanning Fluorimetry

Differential scanning fluorimetry binding assays of selected compounds against TAOK2 were conducted to measure strength of compound binding. A solution of 5 μM TAOK2, 50 mM MgCl2, 50 mM HEPES pH 7.5, and 2.5x SyproOrange(Life Technologies) was made. Compound was added to a final concentration of 1 μM. 25 μl of the reaction solution was added to the wells of a Biorad Multiplate 96 wellclear PCR plate. The plate was covered with a Biorad Microseal ‘B’ seal. Plates were read in a Biorad CFX96 Real-Time PCR system. The temperature was increased from 4° C. to 80° C., with fluorescence measurements being taken every 0.5° C. in the 6-FAM Fluorescein channel. The fluorescence response to heat curve was fit to a binding isotherm in order to determine the inflection point, which was taken as the melt temperature (Tm).

### Radiometric Kinase Assays

Radiometric activity assays of selected compounds against TAOK2 were conducted. A reaction solution of 0.02 mg/ml TAOK2, 50 mM MgCl2, 50 mM HEPES pH 7.5, 1 mg/ml MBP, and increasing compound concentration was made. The compound/DMSO solution was 5% by volume in the final reaction mixture. An ATP solution of 1:20 0.1 μCi/ml 32P -labeled ATP:1 mg/ml ATP was made. 10 μl of ATP solution was added to 50 μl of reaction solution, and allowed to react for 30 minutes. Reactions were stopped by blotting onto filter paper, then submerging in10% trifluoroacetic acid (TFA). Samples were washed 4 times in TFA, then put into scintillation vials. 5 ml Complete Counting Cocktail 3a70B (Research Products International Corp.) was added, and the vials were counted on a Beckman scintillation counter for 10 minutes.

### Chemical Library

The chemical library used for high-throughput screening is comprised of ˜230,000 small molecules small organic molecules. The compounds in the library satisfy a relaxed set of Lipinsky's rules, with 99% having a molecular weight less than 550 (average 250-300). The library also contains approximately 6,500 partially purified natural product fractions isolated from unique marine bacteria. Each natural product fraction contains 3-10 natural products in DMSO and is suitable for high throughput screening.

### Analysis of High Throughput Screening Data

All data was analyzed using the Genedata Screener® software (version 10.1, GeneData, Inc. Basel, Switzerland). For analysis of the data from the primary screen of the chemical library, experimental results obtained from EnVision multi-label plate reader were processed and quality controlled using the Assay Analyzer module of the Genedata Screener® Suite. For each plate, the raw data values for all wells were normalized using equation 1 which assumes hits are infrequent, structurally unrelated, and randomly distributed on individual library plates:

$$NormalizedValues = \frac{RawValues - MedianofTestPopulation}{MedianofTestPopulation} * 100 \qquad ( 1 )$$

The Test Population consists of the chemical library located in columns 3 to 22 of the 384-well assay plate. This assumption was applied to all the library plates except for the natural product collection plates which were normalized to the neutral control (DMSO) instead of the test population. Staurosporine (2.3 μM), a positive control, was included on every assay plate and DMSO was used as a neutral control (columns 2 and 23). Normalized well values were then corrected, where a correction factor each well was calculated using a proprietary pattern detection algorithm in the Assay Analyzer software (See GeneData user documentation and Wu, et al., Journal of Biomolecular Screening, 13:159, 2008). Z-scores were calculated from the corrected normalized activity for each compound.

After cherry picking primary hits, the selected compounds were assayed in a limited dose-response format (10, 3.3, and 1 μM) in triplicate using the primary enzymatic assay for TAOK2. The data for each assay well was normalized to the neutral controls. The normalized activity values (replicates) for each compound in each assay were then condensed to a single value (condensed activity) using the “Robust Condensing” method in Genedata Screener®. The condensed activity is the most representative single value of the triplicates. In general, the triplicates were pre-condensed into a pair of values as follows:

Values(X, Y)=(Median of Triplicates m)÷dispersion (2)

where Dispersion=Median(|X₁−m|, |X₂−m|, |X₃−m|). The less X and Y differ (|X−Y|), the better the data quality. For data points where |X−Y|≧30%, the median of X and Y was used as the condensed activity, which is also the median of the triplicate measurements. Otherwise, a condensing functionMax(X,Y)^{Max(XY)} was used to estimate the condensed activity. A robust Z-Score was then calculated for each compound using equation 3:

$$RobustZ - Score = \frac{CondensedActivity - MedianofNeutralControls}{RobustStandardDeviationofNeutralControls} \qquad ( 3 )$$

### Western Blots

U2OS GFP-LC3 cells were seeded in 6-well plates at 150,000 cells per well in Dulbecco's Modified Eagle Medium supplemented with 10% fetal bovine serum. Twenty-four hours after seeding, the indicated compounds were added to a final concentration of 50 □M, or an equivalent volume of vehicle alone (DMSO) was added. Twenty-four hours after compound addition, cells were rinsed twice with PBS and then lysed in 2× sample buffer. Lysates were boiled for 5 minutes, sonicated to shear DNA, separated by SDS-PAGE, and transferred to PVDF membranes (Biorad). The membranes were blocked in 5% milk in TBS-T (Tris-Buffered-Saline, Tween 20) for one hour at room temperature and then incubated with primary antibody in blocking solution overnight at 4° C. Membranes were then washed, incubated with secondary antibody in blocking solution, washed again, and analyzed by enhanced chemiluminescence followed by exposure to film.

### FACS Analysis

U2OS GFP-LC3 cells were seeded in 6-well plates at 150,000 cells per well in Dulbecco's Modified Eagle Medium supplemented with 10% fetal bovine serum. Twenty-four hours after seeding, the indicated compounds were added to a final concentration of 50 μM, or an equivalent volume of vehicle alone (DMSO) was added. Twenty-four hours after compound addition, cells were rinsed with PBS (Phosphate-Buffered Saline) and analyzed by flow cytometry using a FACS Calibur (BD Biosciences) to measure media GFP fluorescence per cell.

### Example 2

### High-Throughput Screening

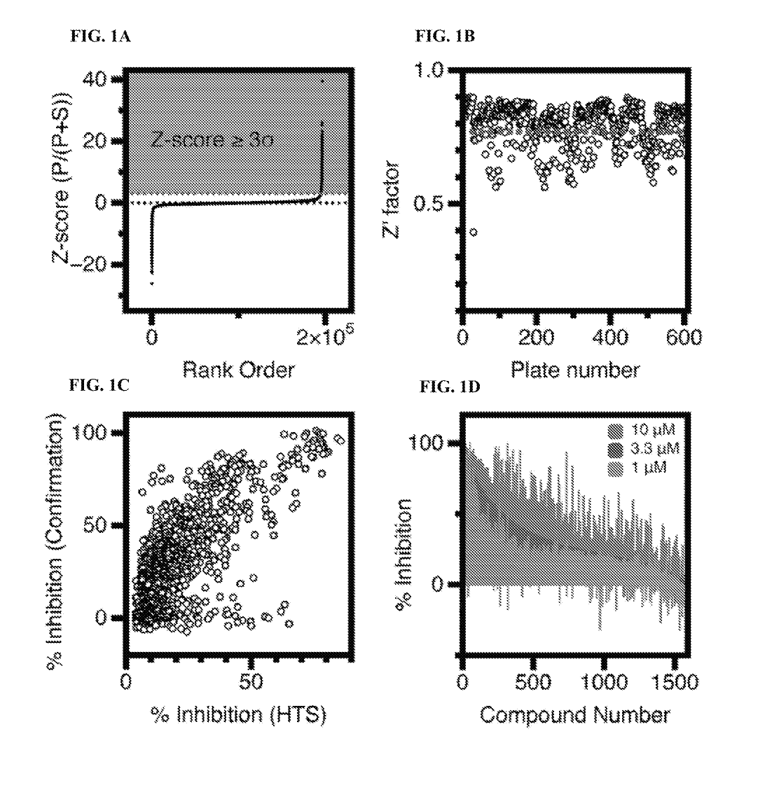

In order to study the relationship between TAOK2 and both cancer and autophagy, a high-throughput screen was performed using a 200,000 compound library to discover novel small-molecule kinase inhibitors (FIG. 1A-D). Phosphorylation reactions were performed in a 384 well format against the generic kinase substrate Myelin Basic Protein (MBP) with the addition of compound. Kinase-Glo (Promega) was then added to each well to detect remaining ATP concentration. Thus, inhibitory compounds would decrease TAOK2 activity, which would increase both remaining ATP concentration and luminescence in those wells.

After the initial screen, 1645 compounds were identified with inhibition greater than three σ above the mean DMSO control (FIG. 1A). The Z′ factor of the initial screen across all plates was 0.77, indicating high significance (FIG. 1B). These compounds were then re-screened in triplicate under the same conditions, with the compounds being assayed at three concentrations (1 μM, 3.3 μM, and 10 μM, FIG. 1C, D). 432 compounds were identified after the re-screen with average inhibitions greater than 10 sigma at the 10 μM concentration. The Z′ factor of the confirmation screen was 0.52, again indicating high significance.

The best 13 compounds from the re-screen are shown in FIG. 2. The prevalence of multiple heterocycles in the compounds is reflective of the composition of the screen (described in Methods in Supplemental Material). These 13 compounds were purchased from Chembridge Inc. and ChemDiv Inc. for measurement of direct binding using Differential Scanning Fluorimetry (DSF). DSF measures protein melt temperature (Tm) with a lipophilic dye (SyproOrange). As the temperature of the solution is increased, measurements of dye fluorescence are taken. When the protein begins to denature, hydrophobic regions in the protein are exposed, and the dye is able to bind. This binding insulates the dye from the surrounding water, reducing quenching effects and allowing fluorescence. Binding of small molecules to protein has a stabilizing effect, and this is seen by an increase in melt temperature (Table 1). The ΔTm values ranged from 3.0 to −0.6° C. The three compounds with the highest positive shifts were SW034538, SW163112, and SW083688, with 3° C., 2.2° C., and 2° C. shifts, respectively.

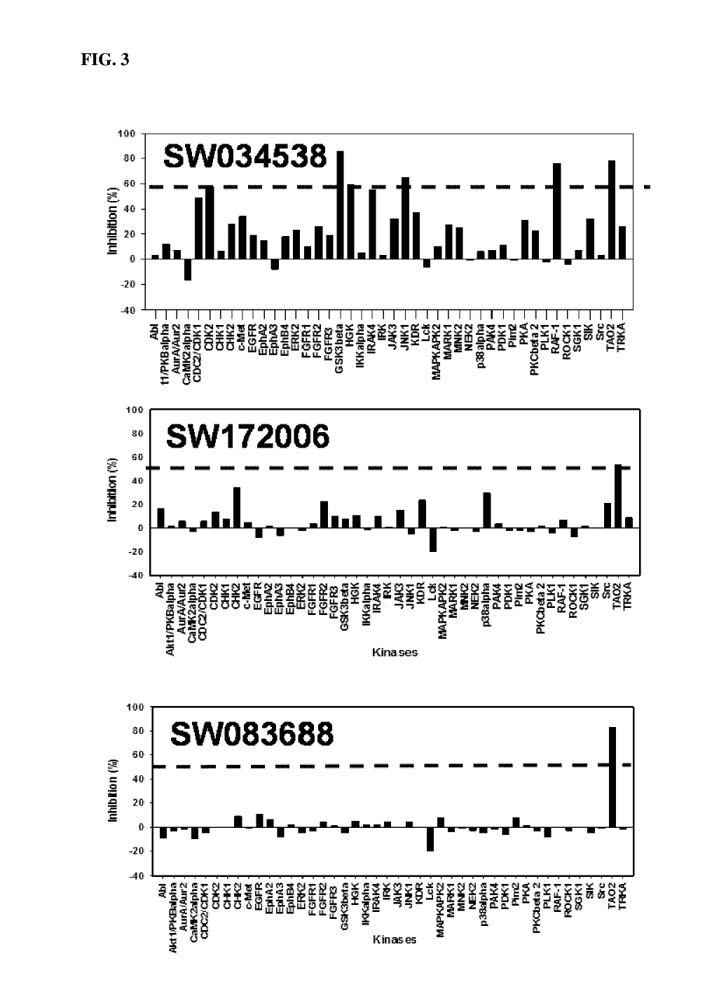

The selectivity of each compound that had a >0.5° C. ΔTm was then determined using DSF against a small panel of other MAPK cascade members (Table 1). The difference between the TAOK2 ΔTm and that of p38 and ASK1 was determined. The three compounds identified as most potent via DSF bound preferentially with TAOK2, though SW083604 had significant cross-reactivity (0.9° C. ΔTm with respect to p38). The majority of the other compounds were also preferentially inhibitory toward TAOK2. Of the 13 compounds, SW034538, SW172006 and SW083688 were cross-verified for selectivity by performing a specificity screen against 45 kinases commercially at Eurofins Cerep, Inc. (FIG. 3). The results show that compounds SW172006 and SW083688 are highly specific toward TAOK2 while compound SW034538 has cross-reactivity with respect to six of the 45 kinases.

### Example 3

### Inhibition of TAOK2 by Newly Identified Compounds

It had previously been determined that TAOK2 is required for autophagic flux in cancer cells. siRNA targeting TAOK2 elicited an expression response similar to siRNA targeting known autophagy genes, such as ULK1, a protein kinase involved in mTOR and AMPK signaling, in the human colon cancer cell line HCT116. TAOK2 depletion also impaired autophagic flux in human osteosarcoma cell line U2OS engineered to stably express the autophagy marker 1A/1B-light chain 3 fused to GFP (GFP-LC3), a microtubule associated protein.

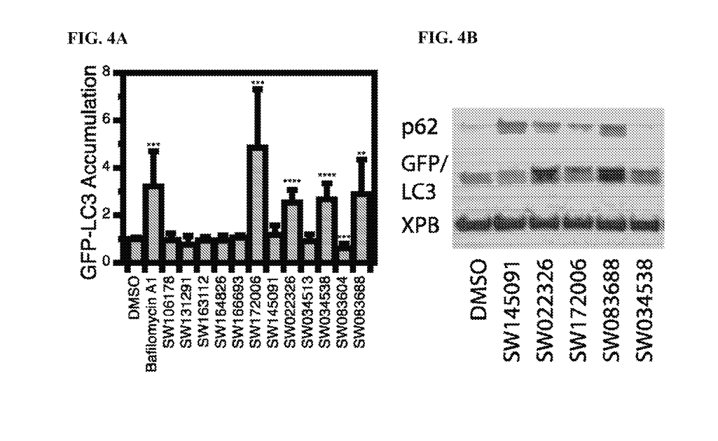

It was investigated whether chemical inhibition of TAOK2 could similarly inhibit autophagy in this cell line. Indeed, four of 13 compounds tested significantly impaired autophagic flux, as indicated by an increase in accumulation of the GFP-LC3 when added to the media at 50 μM concentration for 24 hours (FIG. 4A). Degradation of the autophagy scaffold and LC3 binding protein p62 is a second marker of autophagic flux. p62 degradation was assayed by Western blotting (FIG. 4B). Compounds SW172006, SW022326, and SW083688 each increased the abundance of p62, suggestive of autophagy inhibition. Compound SW034538, despite showing inhibition via GFP-LC3 fusion degradation, did not appear to increase the appearance of p62.

Compounds SW034538 and SW083688 were both selective in the DSF measurements and effective in the GFP-LC3 degradation assays. These compounds were then chosen for radiometric analysis to determine their IC₅₀ values (Table 1). TAOK2 was incubated with inhibitor, MBP, and γ-labeled ATP and allowed to react for 10 minutes. The concentrations of MBP and ATP were chosen such that the reaction was kept in the linear range for the duration of the experiment. The IC₅₀ for compound SW034538 was found to be 300 nM under these conditions, and the IC₅₀ for SW083688 was found to be 1.3 μM. The compounds SW034538 SW172006 and SW083688, which were effective in both the GFP-LC3 assay and were selective for TAOK2 according to DSF, have derivatives in the compound library. These compounds were culled from the 200 k screen computationally to obtain SAR (FIG. 5). IC₅₀ value estimates were calculated from the results of the 200 k rescreen described above. In the case of SW034538 (FIG. 5A), methyl and ethyl amides in the R³ position appeared preferred, with methyl amides having the highest potency (0.08 and 0.2 μM IC₅₀s, respectively). All methyl and ethyl amides in the R³ position that were tested were inhibitive. Bulky hydrophobic groups in this position abolished compound activity. Amines in this position had much weaker binding (>1.7 μM IC₅₀). The R¹, R², and R⁴ positions were broadly tolerant. These results suggest that the thiazole ring containing the R³ moiety forms the greater interaction surface. For SW172006 (FIG. 5B),there is no data for changes in the sulfate group at R¹. On the other hand, R² can be modified with other ring systems. In SW083688 (FIG. 5C), R¹ can be substituted with a flat ring system. R² can be modified with small bulky groups including five- or six- membered rings.

TABLE 1

| | | ΔΔTₘ ° C. | ΔΔTₘ ° C. | | |
|---|---|---|---|---|---|
| Compound | ΔTₘ ° C. | (p38) | (ASK1) | IC₅₀ | |
| SW083604 | 1.2 | 0.3 | 1.1 | 600 | nM |
| SW034538 | 3.0 | 2.8 | 2.7 | 300 | nM* |
| SW083688 | 2.0 | 2.0 | 2.1 | 1.3 | μM* |
| SW022326 | −0.6 | — | — | 4 | μM |
| SW172006 | 1.3 | 2.0 | 1.2 | 5 | μM |
| SW163112 | 2.2 | 1.5 | 1.7 | 3 | μM |
| SW106178 | 1.6 | 1.3 | 1.2 | 6 | μM |
| SW109820 | 0.5 | — | — | 1 | μM |
| SW131291 | 1.2 | 2.0 | −0.0 | 3 | μM |
| SW164826 | −0.2 | — | — | 3 | μM |
| SW166693 | 0.7 | 0.0 | 0.4 | 5 | μM |
| SW145091 | 1.6 | 2.2 | 1.2 | 2 | μM |
| SW034513 | 0.1 | — | — | 3 | μM |

*IC₅₀ determined radiometrically.

Three new, potent, inhibitors of the MAP3K TAOK2 have been identified in this screen. These compounds are not only active in biochemical assays, but are also capable of inhibiting the action of TAOK2 in cells. Use of these inhibitors has revealed TAOK2 is a critical player in autophagy. Previous studies have suggested that TAOK2 may be an anticancer target. The compounds identified in this screen will be vital in further research regarding the function of TAOK2 in cells, as well as provide potential scaffolds for the production of pharmacologically useful anticancer agents.

What is claimed is:

1. A method of treating or preventing a cell proliferative disorder in a subject, comprising administering to said subject a therapeutically sufficient amount of a compound of Formula I: 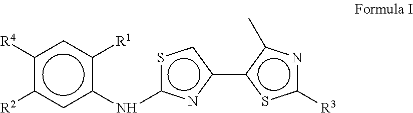
wherein:
R¹ is selected from the group consisting of: OCH₃, OCH₂CH₃, H, CH₃, and CH₂CH₃;
R² is selected from the group consisting of: OCH₃, Cl, H, CH₃, OH, NO₂, COCH₃, and COOH;
R³ is selected from the group consisting of: NHCOCH₂CH₃, NHCOCH₃, NH₂, and NHCOC₆H₅; and
R⁴ is selected from the group consisting of: H, SO₂NH₂, OH, N(CH₃)₂, Br, CH₃, NO₂, and NHCOCH₃.

2. The method of claim 1, wherein R³ is NHCOCH₂CH₃ or NHCOCH₃.

3. The method of claim 1, wherein R³ is NHCOCH₃.

4. The method of claim 1, wherein the compound has Formula II:

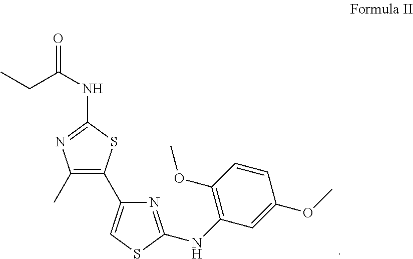 [SMILES: CCC(=O)Nc1nc(C)c(-c2csc(Nc3cc(OC)ccc3OC)n2)s1]

5. The method of claim 1, wherein the cell proliferative disorder is selected from the group consisting of: non-small cell lung cancer or other lung or bronchus cancer, breast cancer, prostate cancer, colon and rectum cancer, bladder cancer, melanoma of the skin, non-Hodgkin lymphoma, thyroid cancer, kidney and renal pelvis cancer, leukemia, endometrial cancer, pancreatic cancer, ovarian cancer, osteosarcoma, and epstein barr virus.

6. The method of claim 1, wherein administering comprises local, regional, systemic, or continual administration.

7. The method of claim 1, wherein said subject is a human.

8. The method of claim 1, further comprising providing to said subject a second therapy.

9. The method of claim 8, wherein said second therapy is provided prior to administering said compound., at the same time as said compound, or after administering said compound.

10. A pharmaceutical composition comprising a compound dispersed in a pharmaceutically acceptable carrier, buffer or diluent, wherein the compound has Formula I: 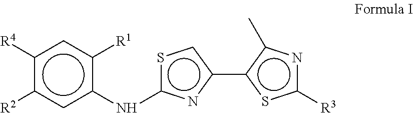
wherein:
R¹ is selected from the group consisting of: OCH₃, OCH₂CH₃, H, CH₃, and CH₂CH₃;
R² is selected from the group consisting of: OCH₃, Cl, H, CH₃, OH, NO₂, COCH₃, and COOH;
R³ is selected from the group consisting of: NHCOCH₂CH₃, NHCOCH₃, NH₂, and NHCOC₆H₅; and
R⁴ is selected from the group consisting of: H, SO₂NH₂, OH, N(CH₃)₂, Br, CH₃, NO₂, and NHCOCH₃.

11. A method of treating or preventing a cell proliferative disorder in a subject, comprising administering to said subject a therapeutically sufficient amount of a compound of Formula III: 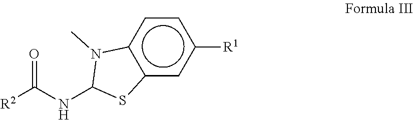
wherein:
R¹ is SO₂CH₃; and
R² comprises a cycloalkyl or aromatic group.

12. The method of claim 11, wherein R² has Formula IV:

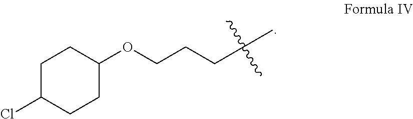 [SMILES: *CCCOC1CCC(Cl)CC1]

13. The method of claim 12, wherein the compound has Formula V:

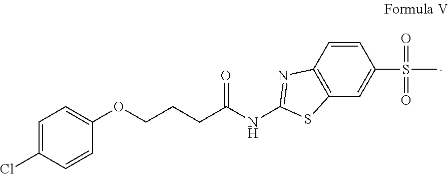 [SMILES: CS(=O)(=O)c1ccc2nc(NC(=O)CCCOc3ccc(Cl)cc3)sc2c1]

14. The method of claim 13, wherein the cell proliferative disorder is selected from the group consisting of: non-small cell lung cancer or other lung or bronchus cancer, breast cancer, prostate cancer, colon and rectum cancer, bladder cancer, melanoma of the skin, non-Hodgkin lymphoma, thyroid cancer, kidney and renal pelvis cancer, leukemia, endometrial cancer, pancreatic cancer, ovarian cancer, osteosarcoma, and epstein barr virus.

15. The method of claim 11, wherein administering comprises local, regional, systemic, or continual administration.

16. The method of claim 11, wherein said subject is a human.

17. The method of claim 11, further comprising providing to said subject a second therapy.

18. The method of claim 17, wherein said second therapy is provided prior to administering said compound., at the same time as said compound, or after administering said compound.

19. A pharmaceutical composition comprising a compound dispersed in a pharmaceutically acceptable carrier, buffer or diluent, wherein the compound has Formula III: 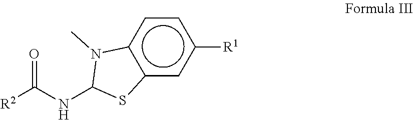
wherein:
R¹ is SO₂CH₃; and
R² comprises a cycloalkyl or aromatic group.

20. A method of treating or preventing a cell proliferative disorder in a subject, comprising administering to said subject a therapeutically sufficient amount of a compound of Formula VI: 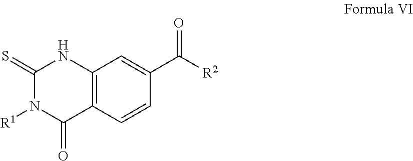
wherein:
R¹ comprises an ether or a flat ring system; and
R² comprises a five- or six-member ring system.

21. The method of claim 22, wherein R¹ has the formula of Formula VII:

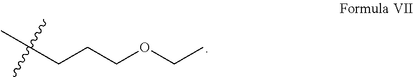 [SMILES: *CCCOCC]

22. The method of claim 22, wherein R² has the formula of Formula VIII:

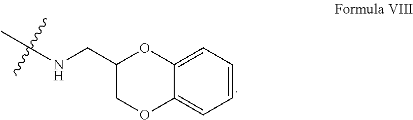 [SMILES: *NCC1COc2ccccc2O1]

23. The method of claim 22, wherein the compound has the formula of Formula IX:

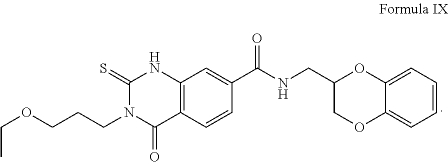 [SMILES: CCOCCCn1c(=S)[nH]c2cc(C(=O)NCC3COc4ccccc4O3)ccc2c1=O]

24. The method of claim 20, wherein the cell proliferative disorder is selected from the group consisting of: non-small cell lung cancer or other lung or bronchus cancer, breast cancer, prostate cancer, colon and rectum cancer, bladder cancer, melanoma of the skin, non-Hodgkin lymphoma, thyroid cancer, kidney and renal pelvis cancer, leukemia, endometrial cancer, pancreatic cancer, ovarian cancer, osteosarcoma, and epstein barr virus.

25. The method of claim 20, wherein administering comprises local, regional, systemic, or continual administration.

26. The method of claim 20, wherein said subject is a human.

27. The method of claim 20, further comprising providing to said subject a second therapy.

28. The method of claim 27, wherein said second therapy is provided prior to administering said compound., at the same time as said compound, or after administering said compound.

29. A pharmaceutical composition comprising a compound dispersed in a pharmaceutically acceptable carrier, buffer or diluent, wherein the compound has Formula III: 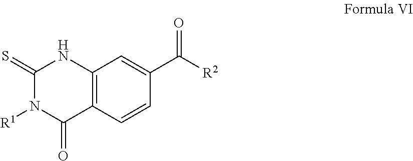
wherein:
R¹ comprises an ether or a flat ring system; and
R² comprises a five- or six-member ring system.
